(19) United States
(12) Patent Application Publication
(10) Pub. No.: US 2019/0046226 A1
(43) Pub. Date: Feb. 14, 2019

(54) ULTRASOUND MEDICAL APPARATUS, ENERGY TREATMENT INSTRUMENT AND CONTROL APPARATUS

(71) Applicant: OLYMPUS CORPORATION, Tokyo (JP)

(72) Inventor: Shinji YASUNAGA, Tokyo (JP)

(73) Assignee: OLYMPUS CORPORATION, Tokyo (JP)

(21) Appl. No.: 16/159,867

(22) Filed: Oct. 15, 2018

Related U.S. Application Data
(63) Continuation of application No. PCT/JP2016/063661, filed on May 6, 2016.

(51) Int. Cl. A61B 17/32; A61B 17/3205

(52) U.S. Cl. CPC A61B 17/320092; A61B 2017/320093; A61B 17/3205

(57) ABSTRACT

An ultrasound medical apparatus includes an ultrasound transducer configured to generate ultrasound vibration, a vibration transmitting member including a contact surface configured to contact a biological tissue, a high heat conductive member placed in a center of the contact surface, and a low heat conductive member placed around the high heat conductive member, and configured to transmit the ultrasound vibration to the biological tissue, and a control apparatus configured to control drive of the ultrasound transducer to a first mode of driving at a first resonance frequency of the ultrasound transducer, and a second mode of driving at a second resonance frequency of a structure provided with the ultrasound transducer and the vibration transmitting member.

### CROSS REFERENCE TO RELATED APPLICATION

This application is a continuation application of PCT/JP2016/063661 filed on May 6, 2016, the entire contents of which are incorporated herein by this reference.

### BACKGROUND OF THE INVENTION

### Field of the Invention

The present invention relates to an ultrasound medical apparatus including an ultrasound transducer, used in an endoscopic surgical operation, and grips a biological tissue to perform joining/cutoff and the like, an energy treatment instrument and a control apparatus.

### Description of the Related Art

In recent years, a treatment instrument that joins/cuts off a biological tissue by using energy in an endoscopic surgical operation, for example, has been developed. In the treatment instruments like this, a treatment instrument using energy by ultrasound is known.

For example, Japanese Patent Application Laid-Open Publication No. 2011-92727 discloses an apparatus for sealing a biological tissue, which is an energy treatment instrument using ultrasound.

Japanese Patent Application Laid-Open Publication No. 2011-92727 discloses a technique of having a transducer that generates ultrasound in a jaw configured to be a treatment section, performing joining by giving ultrasound to a biological tissue, and cutting off the biological tissue by a cutter after the biological tissue is joined.

That is, the conventional energy treatment instrument includes an ultrasound transducer in a jaw provided at a distal end of an insertion portion, and is configured to cut off biological tissue by a cutter after joining of the biological tissue.

### SUMMARY OF THE INVENTION

An ultrasound medical apparatus of one aspect of the present invention includes an ultrasound transducer configured to generate ultrasound vibration, a vibration transmitting member including a contact surface configured to contact a biological tissue, a high heat conductive member placed in a center of the contact surface, and a low heat conductive member placed around the high heat conductive member, and configured to transmit the ultrasound vibration to the biological tissue, and a control apparatus configured to control drive of the ultrasound transducer to a first mode of driving at a first resonance frequency of the ultrasound transducer, and a second mode of driving at a second resonance frequency of a structure provided with the ultrasound transducer and the vibration transmitting member.

An energy treatment instrument of one aspect of the present invention includes an ultrasound transducer configured to generate ultrasound vibration, a vibration transmitting member including a contact surface configured to contact a biological tissue, a high heat conductive member placed in a center of the contact surface, and a low heat conductive member placed around the high heat conductive member, and configured to transmit the ultrasound vibration to the biological tissue, and a switch configured to operate a first mode of driving the ultrasound transducer at a first resonance frequency of the ultrasound transducer, and a second mode of driving the ultrasound transducer at a second resonance frequency of a structure provided with the ultrasound transducer and the vibration transmitting member.

A control apparatus of one aspect of the present invention includes a control unit configured to output to an ultrasound transducer, a first control signal to drive at a first resonance frequency of the ultrasound transducer configured to generate ultrasound vibration, and a second control signal to drive at a second resonance frequency of a structure provided with the ultrasound transducer and a vibration member configured to transmit the ultrasound vibration to a biological tissue.

### BRIEF DESCRIPTION OF THE DRAWINGS

FIG. 1 is a perspective view illustrating an entire configuration of an ultrasound medical apparatus of one aspect of the present invention;

FIG. 2 is a perspective view illustrating a configuration of a grasping portion of an energy treatment instrument of one aspect of the present invention;

FIG. 3 is a sectional view illustrating the configuration of the grasping portion of one aspect of the present invention;

FIG. 4 is an exploded perspective view illustrating the configuration of the grasping portion of one aspect of the present invention;

FIG. 5 is a perspective view illustrating the configuration of the grasping portion of one aspect of the present invention;

FIG. 6 is a schematic view illustrating the grasping portion and a control apparatus of one aspect of the present invention, and including a section illustrating a state in which a biological tissue is grasped;

FIG. 7 is a schematic view illustrating the grasping portion and the control apparatus of one aspect of the present invention, and including a section illustrating a state of joining the biological tissue; and

FIG. 8 is a schematic view illustrating the grasping portion and the control apparatus of one aspect of the present invention, and including a section illustrating a state of cutting off the biological tissue.

### DETAILED DESCRIPTION OF THE PREFERRED EMBODIMENT

Hereinafter, the present invention will be described with use of the drawings.

Note that in the following explanation, drawings based on each embodiment are schematic. Attention should be paid to the fact that relationships between thicknesses and widths of respective portions, ratios of the thicknesses of the respective portions and the like are different from the actual relationships, ratios and the like. Among the drawings, the parts where mutual dimensional relationships and ratios are different may be included.

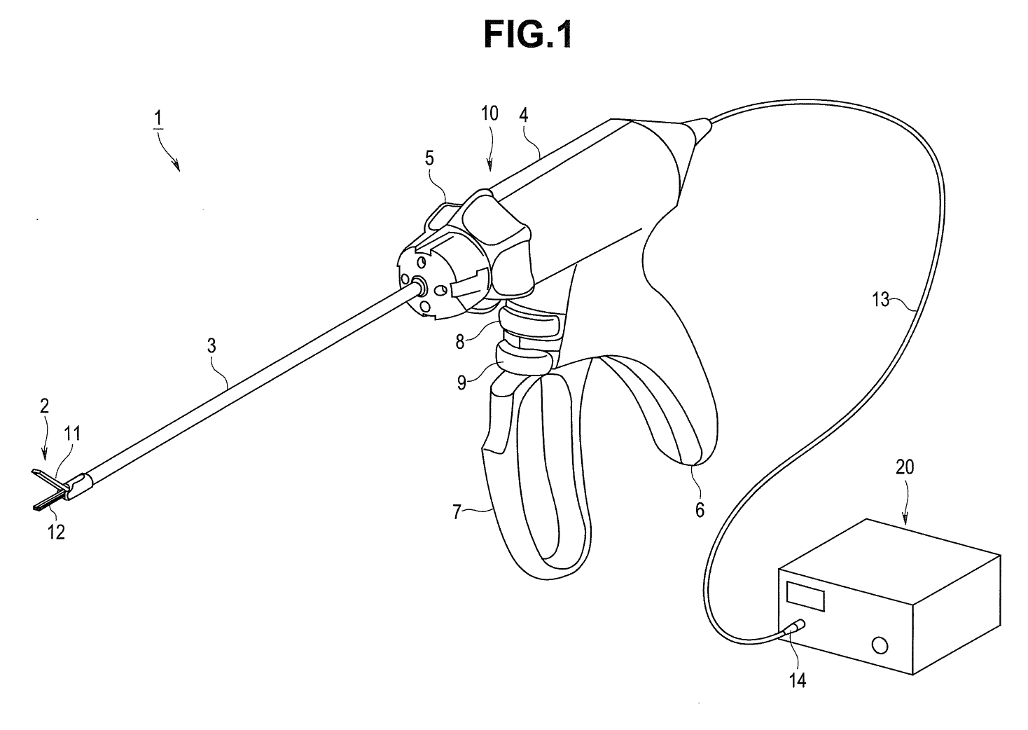

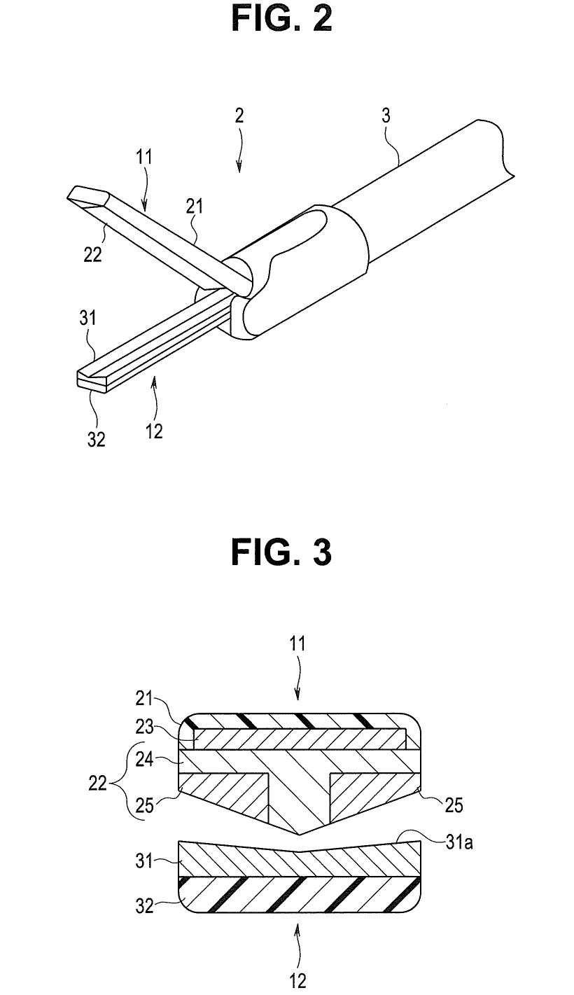

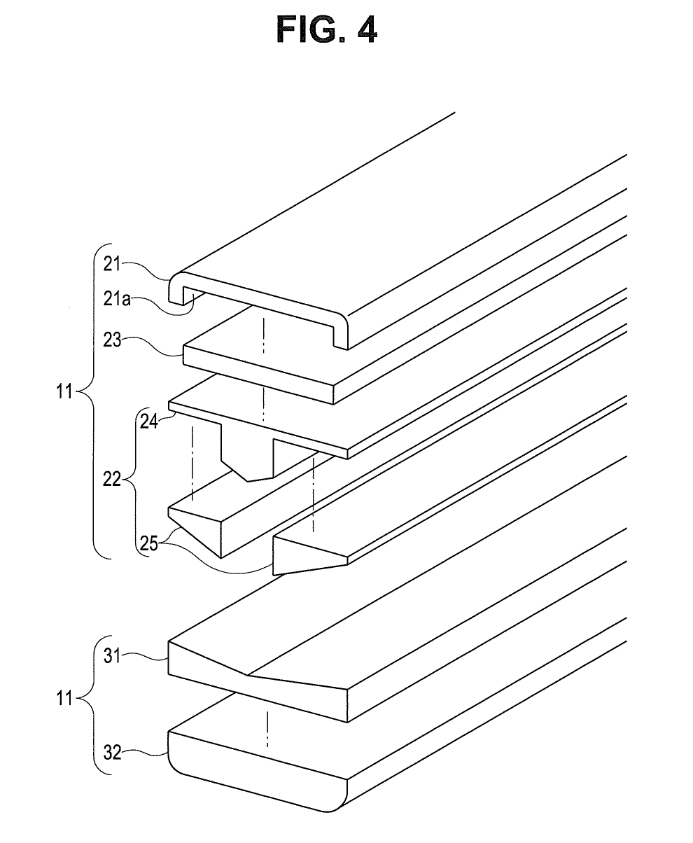

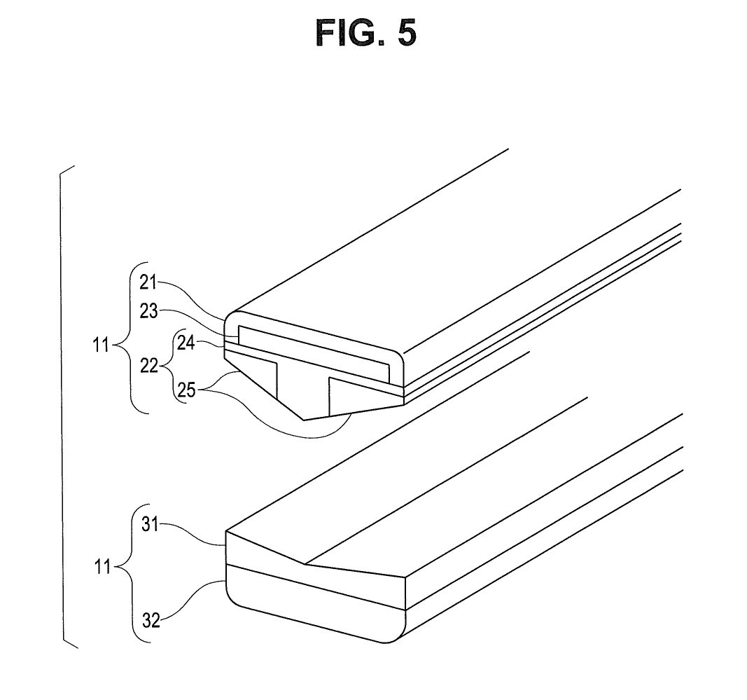

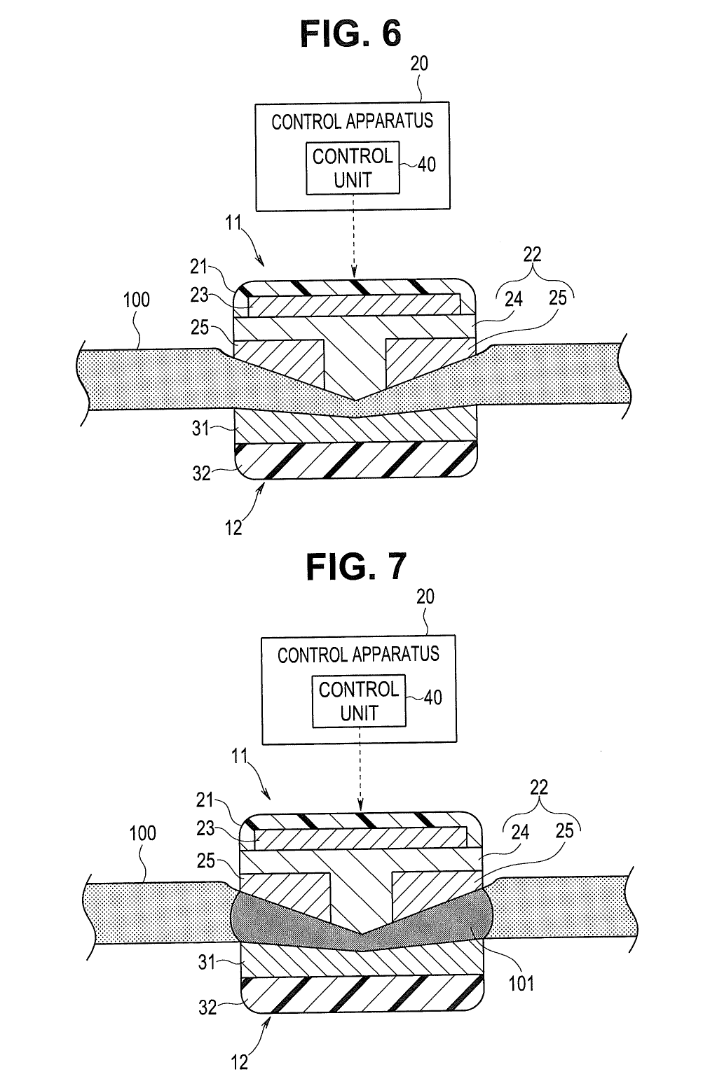

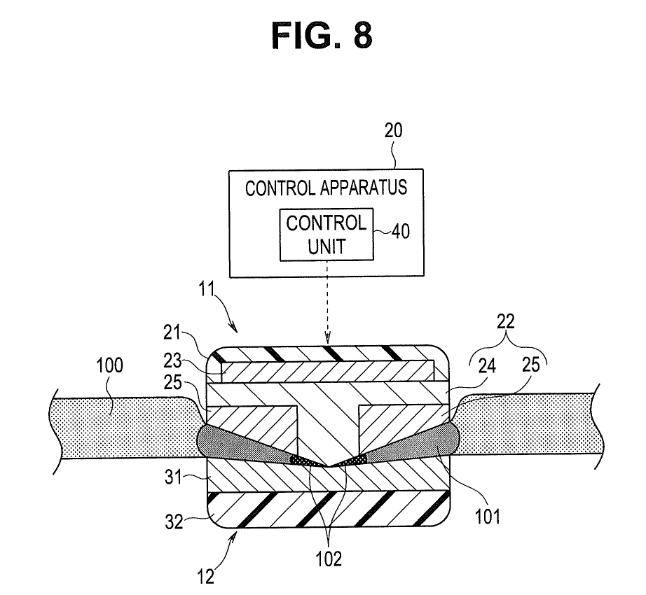

FIG. 1 is a perspective view illustrating an entire configuration of an ultrasound medical apparatus of one aspect of the present invention. FIG. 2 is a perspective view illustrating a configuration of a grasping portion of an energy treatment instrument. FIG. 3 is a sectional view illustrating the configuration of the grasping portion. FIG. 4 is an exploded perspective view illustrating the configuration of the grasping portion. FIG. 5 is a perspective view illustrating the configuration of the grasping portion. FIG. 6 is a schematic view illustrating the grasping portion and a control apparatus, and including a section illustrating a state in which a biological tissue is grasped. FIG. 7 is a schematic view illustrating the grasping portion and the control apparatus, and including a section illustrating a state of joining biological tissue. FIG. 8 is a schematic view illustrating the grasping portion and the control apparatus, and including a section illustrating a state of cutting off the biological tissue.

### (Ultrasound Medical Apparatus)

First, the configuration of the ultrasound medical system of a present embodiment will be described hereinafter.

As illustrated in FIG. 1, an ultrasound medical apparatus 1 which is an ultrasound system is configured by including an energy treatment instrument 10 which is an ultrasound treatment instrument, and a control apparatus 20 which is a processor.

The energy treatment instrument 10 which is the ultrasound treatment instrument as a surgical operation device is configured by including a grasping portion 2 configured to be a probe distal end portion grasping a biological tissue to perform joining (seal)/cutoff (dissection) and the like as a treatment section, an insertion tube portion 3, and an operation portion 4 in order from a distal end.

The grasping portion 2 has an upper jaw 11 as an ultrasound blade (ultrasound probe), and a substantially plate-shaped lower jaw 12 formed of a material having biocompatibility. The upper jaw 11 is provided to be openable and closable to the lower jaw 12.

Here, in the present embodiment, the upper jaw 11 is an ultrasound blade, but the lower jaw 12 may be an ultrasound blade.

The insertion tube portion 3 provided with the grasping portion 2 is configured with a rigid metal pipe. The insertion tube portion 3 is configured to be rotatable so as to oscillate laterally in a range of an approximately half rotation (180°) around a longitudinal axis by rotationally operating a rotation operation member 5 connected and fixed to a proximal end portion from a neutral position.

Thereby, the grasping portion 2 is rotated around the longitudinal axis of the insertion tube portion 3 in association with rotation of the insertion tube portion 3. That is, the ultrasound medical apparatus 1 in this case is configured to be able to oscillate the grasping portion 2 in a direction in which the grasping portion 2 easily grasps a biological tissue, by the rotation operation of the rotation operation member 5.

The operation portion 4 is configured by including the aforementioned rotation operation member 5 configured to rotationally operate the insertion tube portion 3, a fixed handle 6 for holding at a time of use, a movable handle 7 configured to perform an opening and closing operation of the upper jaw 11, a first handle switch 8 configured to operate a first mode of performing joining and the like of a biological tissue such as a blood vessel, and a second handle switch 9 configured to operate a second mode of performing cutoff and the like of a biological tissue.

Note that a cable 13 for electric power supply is connected to the operation portion 4. The cable 13 has a connector 14 provided at an end portion. The connector 14 is detachably connected to the control apparatus 20.

Further, the energy treatment instrument 10 may be configured such that a bending portion is provided at a portion behind the grasping portion 2, of the insertion tube portion 3, and an operation member configured to perform a bending operation of the bending portion is provided at the operation portion 4.

According to the above explanation, the ultrasound medical apparatus 1 of the present embodiment configures ultrasound coagulation dissection forceps configured to grasp a biological tissue by the grasping portion 2 of the energy treatment instrument 10 connected to the control apparatus 20 to perform joining/cutoff and the like.

### (Grasping Portion)

Next, a configuration of the grasping portion 2 of the present embodiment will be described hereinafter.

As illustrated in FIG. 2, the upper jaw 11 of the grasping portion 2 has an upper cover 21 formed of a heat-resistant resin having biocompatibility, and a blade 22 as a vibration transmitting member that is exposed from a lower end of the upper cover 21 and has a pentagonal sectional shape in this case, and a transducer 23 illustrated in FIG. 3 is incorporated in the upper cover 21.

The lower jaw 12 of the grasping portion 2 has a lower grasping member 31 formed of a heat-resistant resin, ceramics or the like, and a lower cover 32 placed to cover an undersurface of the lower grasping member 31 and formed of a heat-resistant resin having biocompatibility.

Note that the upper cover 21 and the lower cover 32 of the grasping portion 2 are desirably formed of a heat-resistant resin member such as PEEK, PFA or LCP (liquid crystal polymer) to prevent an unexpected heat damage of a peripheral tissue caused by high temperature of the upper jaw 11 and the lower jaw 12 accompanying treatment of a biological tissue.

As illustrated in FIG. 3 to FIG. 5, the blade 22 of the upper jaw 11 is configured by including a high heat conductive vibration transmitting member 24 that is a high heat conductive member, and two low heat conductive vibration transmitting members 25 that are low heat conductive members.

The high heat conductive vibration transmitting member 24 is formed of copper, aluminum nitride or the like, and is a plate-shaped block member substantially T-shaped in section.

The two low heat conductive vibration transmitting members 25 are formed of zirconium, a titanium alloy or the like, and are block members trapezoidal in section, which are joined to both side portions of a lower part of the high heat conductive vibration transmitting member 24 by brazing and soldering, bonding by a heat-resistant resin or the like.

Note that it does not matter whether the high heat conductive vibration transmitting member 24 and the low heat conductive vibration transmitting member 25 are electrically conductive or insulative, but it is desirable that the high heat conductive vibration transmitting member 24 and the low heat conductive vibration transmitting member 25 are insulating members in consideration of the electrical configuration as the treatment instrument that treats a biological tissue.

The two low heat conductive vibration transmitting members 25 are joined to the high heat conductive vibration transmitting member 24, and thereby the blade 22 is configured with a pentagonal sectional shape.

Note that as for the shape of the blade 22, an example configured with a pentagonal section, for example, is shown in the present embodiment, but a configuration in which ultrasound vibration from the transducer 23 can be at least transmitted to the target biological tissue can be adopted.

When cutoff performance for a biological tissue is made better, it is desirable in terms of efficiency that a polygon including a shape in which a central portion to be a portion that cuts off a biological tissue 100 is protruded, contacts the biological tissue, in a contact surface where the section of the blade 22 contacts the biological tissue 100.

Therefore, the blade 22 of the present embodiment is provided in such a manner that a side of a shape in which the section to be the contact surface contacting a biological tissue is protruded faces the lower jaw 12. Further, when importance is placed on joining performance of the biological tissue, the contact surface with a biological tissue may be a plane, that is, a sectional shape of the blade 22 may be rectangular.

In the blade 22 configured in this way, the plate-shaped transducer 23 using a piezoelectric single crystal element such as lithium niobate single crystal (LiNbO3) in this case is joined to a top surface side of the high heat conductive vibration transmitting member 24. The upper cover 21 is placed to cover the transducer 23 and the upper jaw 11 of the grasping portion 2 is configured.

A recessed portion 21a (refer to FIG. 4) in a similar shape to the transducer 23 is formed in the upper cover 21, and is bonded to the transducer 23 by an adhesive of a heat-resistant resin or the like to cover the transducer 23 without gaps.

The transducer 23 is an ultrasound transducer having a piezoelectric crystal element, electrodes, metal mass and the like are stacked on one another, and configured to vibrate in two modes by a control signal outputted from the control apparatus 20 by a control cable inserted through the cable 13 being connected to the ultrasound transducer.

As a piezoelectric material of the transducer 23 in this case, lithium niobate single crystal (LiNbO3) of piezoelectric single crystal having a Curie point of approximately 1200° C., in which piezoelectric characteristics are hardly deteriorated with progress of use, at a time of joining, is used.

Further, the lithium niobate single crystal (LiNbO3) is one of lead-free piezoelectric materials having a high mechanical Q value suitable to an ultrasound transducer for high output applications, uses no lead, and therefore is excellent in environmental friendliness. As for the material used in the transducer in the present embodiment, such a material is used that has a Curie point a half of which or less corresponds to a maximum treatment temperature by the energy treatment instrument 10.

Note that the piezoelectric single crystal material used in the transducer 23 may be a piezoelectric single crystal material such as a lithium tantalate (LiTaO3) without being limited to lithium niobate single crystal (LiNbO3).

The lower grasping member 31 of the lower jaw 12 is a plate-shaped member in which a top surface 31a facing the blade 22 of the upper jaw 11 inclines toward a center. The lower grasping member 31 is desirably formed of a heat-resistant resin with low heat conduction, ceramics or the like to restrain diffusion of heat at a time of cutoff of a biological tissue. The lower cover 32 is bonded to an undersurface of the lower grasping member 31 by an adhesive of a heat-resistant resin or the like, and the lower jaw 12 is configured.

The ultrasound medical apparatus 1 of the present embodiment configured as above sandwiches a biological tissue such as a blood vessel with the grasping portion 2 of the energy treatment instrument 10, drives the transducer 23 of the upper jaw 11 and can perform joining, cutoff and the like of the biological tissue by heat and vibration.

### (Operation)

Here, operations of performing joining, cutoff and the like of the biological tissue 100 such as a blood vessel by the ultrasound medical apparatus 1 will be described hereinafter.

First, as illustrated in FIG. 6, in the grasping portion 2 of the energy treatment instrument 10, the biological tissue 100 is sandwiched by the upper jaw 11 and the lower jaw 12, whereby the biological tissue 100 is grasped.

At this time, the grasping portion 2 in this case has a structure in which a pressing force in a central portion of the biological tissue 100 grasped by the grasping portion 2 is larger than pressing forces in both end portions, due to the sectional shape of the contact surface which the blade 22 of the upper jaw 11 contacts.

By operation of the first handle switch 8 of the operation portion 4, as the first mode, the control unit 40 of the control apparatus 20 applies a control signal of a predetermined voltage that drives the transducer 23 at a high frequency in an MHz band, for example, of 10 to 30 MHz which corresponds to a resonance frequency of the transducer itself, to the transducer 23 provided in the upper jaw 11.

At this time, the transducer 23 generates ultrasound vibration of 10 MHz to 30 MHz, and causes the biological tissue 100 to propagate/absorb vibration at the high frequency via the high heat conductive vibration transmitting member 24.

By using the very high frequency like this, it becomes possible to heat a joint portion with relatively small energy and obtain high sealing strength without giving a large heat damage, because the ultrasound energy is absorbed inside the biological tissue 100 and generates heat.

Thereby, as illustrated in FIG. 7, the biological tissue 100 in a region grasped by the grasping portion 2 is heated and sealed, and a joint site 101 is formed.

Note that the joint site 101 can be formed with a tissue temperature in a grasped region of the biological tissue 100 by the grasping portion 2 being approximately 200° C., and a treatment time period of approximately 10 to 20 seconds.

Further, the transducer 23 in this case has the piezoelectric single crystal of lithium niobate single crystal (LiNbO3), and a resonance frequency in a thickness direction is 30 MHz to a thickness of approximately 0.1 mm, so that by making the thickness of the transducer 23 0.1 mm to 0.3 mm, for example, the driving frequency becomes a high frequency of 10 MHz to 30 MHz.

In this way, the first mode of joining the biological tissue 100 grasped by the grasping portion 2 of the transducer 23 is a mode in which the transducer 23 is driven at the resonance frequency of itself.

Next, by operation of the second handle switch 9 of the operation portion 4, as the second mode, the control unit 40 of the control apparatus 20 applies to the transducer 23, a control signal of a predetermined voltage that is higher than the voltage in the first mode, and drives the transducer 23 at a high frequency in a kHz band, for example, of 150 kHz which corresponds to a resonance frequency of the entire upper jaw 11.

Heat that is generated at this time is transmitted through the high heat conductive vibration transmitting member 24, the joint site 101 of the biological tissue 100 grasped by the grasping portion 2 is heated, and frictional heat on the tissue surface caused by vibration of the entire upper jaw 11 also contributes to heating of the joint site 101.

Thereby, as illustrated in FIG. 8, the joint site 101 is heated to a high temperature in a short time period, disintegration of the tissue occurs, and a vicinity (cutoff site 102) of the center of the biological tissue 100 grasped by the grasping portion 2 especially with a large pressing force is cut off.

Here, both end portions of the grasped joint site 101 of the biological tissue 100 are restrained from being heated due to existence of the low heat conductive vibration transmitting members 25 with a low thermal conductivity, so that heat invasion in the joint site 101 and a peripheral site of the joint site 101 can be reduced.

Note that the cutoff site 102 can be formed with a treatment time period of approximately one to three seconds of the tissue temperature in the joint site 101 of the biological tissue 100, to which heat is transmitted through the high heat conductive vibration transmitting member 24, of approximately 300° C.

Further, in this case, a higher voltage (higher power) than in the first mode is applied to the transducer 23, and the transducer 23 is driven at a high frequency in a kHz band, 150 kHz in this case, which corresponds to the resonance frequency of the upper jaw 11.

In this way, the second mode of cutting off the biological tissue 100 grasped by the grasping portion 2 of the transducer 23 is a mode in which the transducer 23 is driven at the resonance frequency of the upper jaw 11.

While in the above description, the configuration is illustrated, in which the first mode of joining the biological tissue 100 grasped by the grasping portion 2 of the transducer 23 and the second mode of cutting off the biological tissue 100 are switched by operation of the first handle switch 8 and the second handle switch 9 of the operation portion 4, a configuration can be also adopted, in which the first mode and the second mode can be continuously carried out by control by the control unit 40 of the control apparatus 20 by operation of one switch.

The ultrasound medical apparatus 1 of the present embodiment configured as above does not require a mechanical cutter cutoff mechanism to cut off a biological tissue as in the conventional ultrasound medical apparatus, and can reduce size and cost of the energy treatment instrument 10.

Accordingly, the ultrasound medical apparatus 1 can carry out joining and cutoff of the biological tissue 100 by the grasping portion 2 of the energy treatment instrument 10 as a same component member, and has an advantage of reducing size and cost.

Furthermore, the ultrasound medical apparatus 1 can perform treatment of joining and cutoff by one action by grasping the biological tissue 100 with the grasping portion 2 by the energy treatment instrument 10, and does not require the operation of cutting off the biological tissue 100 by the cutter mechanism of the grasping portion 2, which is added to the operation of grasping and joining the biological tissue 100, as in the conventional ultrasound medical apparatus.

Further, the energy treatment instrument 10 is configured so that the characteristics of the energy treatment instrument 10 are not deteriorated even when the energy treatment instrument 10 is heated to the temperature required at the time of joining and cutting off the biological tissue 100, by using in the transducer 23, the piezoelectric single crystal of lithium niobate single crystal (LiNbO3) with a high Curie temperature of approximately 1200° C. instead of lead zirconate titanate (PZT) that has been widely used conventionally.

Furthermore, the ultrasound medical apparatus 1 has the first mode of controlling drive of the transducer 23 to high frequency drive at the resonance frequency of the transducer 23 that heats by propagation/absorption of ultrasound to the biological tissue 100 with low power by the control apparatus 20, and heating and joining an entire range of the grasped region of the biological tissue 100, and the second mode of controlling the drive of the transducer 23 to high frequency (lower frequency as compared with the first mode) drive of the resonance frequency of the structure of the entire upper jaw 11 that heats by propagation/absorption of ultrasound to the biological tissue 100 with large power, locally increasing a temperature of the vicinity of the center of the grasped region of the biological tissue 100, and cutting off the vicinity of the center of the grasped region with the frictional heat caused by vibration of the upper jaw 11 also working.

In particular, in the second mode, the blade 22 is such that the low heat conductive vibration transmitting members 25 are placed at both sides of the high heat conductive vibration transmitting member 24, and the vicinity of the center of the grasped region of the biological tissue 100 can be locally increased in temperature, so that heat invasion in regions other than the cutoff (dissection) region of the biological tissue 100 can be restrained.

The invention described above is not limited to the embodiment and modifications, and besides the embodiment and modifications, various modifications can be carried out within the range without departing from the gist of the present invention in the stage of carrying out the invention. Further, the above described embodiment includes the inventions in the various stages, and various inventions can be extracted by arbitrary combinations in the plurality of components which are disclosed.

For example, even when several components are deleted from all of the components shown in the embodiment, the configuration from which these components are deleted can be extracted as the invention if the problem that is described can be solved and the effect that is described can be obtained.

What is claimed is:

1. An ultrasound medical apparatus comprising:
an ultrasound transducer configured to generate ultrasound vibration;
a vibration transmitting member including a contact surface configured to contact a biological tissue, a high heat conductive member placed in a center of the contact surface, and a low heat conductive member placed around the high heat conductive member, and configured to transmit the ultrasound vibration to the biological tissue; and
a control apparatus configured to control drive of the ultrasound transducer to a first mode of driving at a first resonance frequency of the ultrasound transducer, and a second mode of driving at a second resonance frequency of a structure provided with the ultrasound transducer and the vibration transmitting member.

2. The ultrasound medical apparatus according to claim 1,
wherein the first resonance frequency is a resonance frequency that joins the biological tissue, and
the second resonance frequency is a frequency lower than the first resonance frequency, and cuts off the biological tissue.

3. The ultrasound medical apparatus according to claim 1,
wherein the ultrasound transducer is configured of a material of piezoelectric crystal.

4. The ultrasound medical apparatus according to claim 1,
wherein in the vibration transmitting member, the high heat conductive member is longer than the low heat conductive member in a distance from the ultrasound transducer to the contact surface.

5. The ultrasound medical apparatus according to claim 2,
wherein the first resonance frequency is in a MHz band, and
the second resonance frequency is in a kHz band.

6. An energy treatment instrument comprising:
an ultrasound transducer configured to generate ultrasound vibration;
a vibration transmitting member including a contact surface configured to contact a biological tissue, a high heat conductive member placed in a center of the contact surface, and a low heat conductive member placed around the high heat conductive member, and configured to transmit the ultrasound vibration to the biological tissue; and
a switch configured to operate a first mode of driving the ultrasound transducer at a first resonance frequency of the ultrasound transducer, and a second mode of driving the ultrasound transducer at a second resonance frequency of a structure provided with the ultrasound transducer and the vibration transmitting member.

7. A control apparatus comprising:
a control unit configured to output to an ultrasound transducer,
a first control signal to drive at a first resonance frequency of the ultrasound transducer configured to generate ultrasound vibration, and
a second control signal to drive at a second resonance frequency of a structure provided with the ultrasound transducer and a vibration member configured to transmit the ultrasound vibration to a biological tissue.
